(19) United States
(12) Patent Application Publication
(10) Pub. No.: US 2026/0026853 A1
(43) Pub. Date: Jan. 29, 2026

(54) DEVICE FOR CHEST WALL RECONSTRUCTION

(71) Applicant: Jeko Metodiev MADJAROV, Wilmington, NC (US)

(72) Inventors: Jeko Metodiev MADJAROV, Wilmington, NC (US); Sophia Jekova MADJAROVA, Wilmington, NC (US)

(21) Appl. No.: 19/278,014

(22) Filed: Jul. 23, 2025

Related U.S. Application Data
(60) Provisional application No. 63/674,514, filed on Jul. 23, 2024.

(51) Int. Cl. A61B 17/80; A61B 17/56

(52) U.S. Cl. CPC A61B 17/8076; A61B 17/8085; A61B 2017/564

(57) ABSTRACT

Some embodiments of the present disclosure are directed to devices for repair of the chest wall and associated methods. Embodiments of the device include a reinforcement member and a body member surrounding and supporting the reinforcement member. The reinforcement member may define at least one opining. The device may be designed to be placed on an outer surface of bone near an injury or defect, and each opening may be designed to receive a fastener so as to secure the device to the bone at the location to be repaired. When installed, the injury or defect may be substantially stabilized to allow healing to occur. In this way, embodiments of the device and method provide a modular device for soft tissue or structural fixation, graft support, or reinforcement in medical applications.

### CROSS-REFERENCE TO RELATED APPLICATIONS

This application claims priority to U.S. Application No. 63/674,514, titled Woven Endoplate for Sternal Reconstruction, filed Jul. 23, 2024, the content of which is hereby incorporated by reference in its entirety.

### TECHNOLOGICAL FIELD

The present disclosure relates in general to devices and methods for sternal reconstruction and chest wall repair.

### BACKGROUND

When a person experiences a thoracic event, sternal/chest wall reconstruction or repair may be needed. A thoracic event could involve trauma to the thoracic cavity or disease of the chest wall. Following the thoracic event, the sternum is typically opened, allowing a surgeon to reach the organs behind the sternum and ribs of the patient, such as occurs during open heart surgery. The sternum is then reconstructed in order to close the sternum along the incision.

Following thoracic surgery, the chest wall may become unstable. In some cases, instability is due to bone that has not healed from thoracic surgery.

### GENERAL DESCRIPTION

Accordingly, embodiments of the invention described herein offer adaptability, secure fixation, and biocompatibility by introducing a composite design that combines structural rigidity and material flexibility. In particular, a modular device is described for soft tissue or structural fixation, graft support, or reinforcement in medical applications. Embodiments provide a hybrid anchoring mechanism combining a rigid support feature with a flexible fabric interface and/or an integrated channel for multiple fixation options.

In some embodiments, a device is provided comprising a reinforcement member and a body member surrounding and supporting the reinforcement member. The reinforcement member may define at least one opening. The device may be configured to be placed on an outer surface of bone proximate an injury or defect. Each opening of the reinforcement member may be configured to receive a fastener so as to secure the device to the bone. When installed, the injury or defect may be substantially stabilized to allow healing to occur.

In some cases, the reinforcement member may comprise a plurality of grommets. An inner surface of at least one grommet may define threads configured to engage corresponding threads on the fastener. The grommets may be arranged linearly or in a z-formation or in any other pattern or arrangement. In some cases, the grommets may be interconnected to form a plate.

In some embodiments, the body member may be a woven material. Moreover, the body member may comprise a channel proximate an edge of the body member that is configured to allow a guidewire to pass therethrough. The channel may, in some cases, be a tube attached to the body member. In other cases, the channel may be a compartment formed by the body member.

The body member may comprise elongated ends.

In some embodiments, the reinforcement member may be integral to the body member. Additionally or alternatively, the body member may define a first longitudinal edge and a second longitudinal edge opposite the first longitudinal edge. A thickness of the body member may decrease from the first longitudinal edge toward the second longitudinal edge. Moreover, the body member may define a channel proximate the first longitudinal edge configured to allow a wire to pass therethrough.

In some embodiments, the reinforcement member may comprise at least one of metal, a bio-polymer, polyetheretherketone (PEEK), or polyetherketoneketone (PEKK). The body member may comprise at least one of polytetrafluoroethylene (ePTFE), expanded polyethylene (cPE), decellularized fish skin, acellular dermal material, biocellular material, collagen, or porcine small intestinal submucosa (SIS). At least one edge of the body member may be contoured.

In other embodiments, a method for chest wall repair is provided. The method may include providing a device comprising a reinforcement member and a body member surrounding and supporting the reinforcement member, where the reinforcement member defines at least one opening, and where the body member comprises a channel proximate an edge of the body member and configured to allow a guidewire to pass therethrough. The method may further comprise inserting the guidewire through an incision and anchoring the guidewire at a site of an injury or defect. The guidewire may be passed through the channel, and the device may be slid along the guidewire to the site of the injury or defect. The method may further include applying at least one fastener to the device via the at least one opening of the reinforcement member to affix the device at the site of the injury or defect.

In some embodiments, the device may be a first device, the reinforcement member may be a first reinforcement member, the body member may be a first body member, the opening may be a first opening, and the channel may be a first channel. A second device may be provided comprising a second reinforcement member, where the second reinforcement member defines at least one second opening. The second device may further comprise a second body member surrounding and supporting the second reinforcement member, where the second body member comprises a second channel proximate an edge of the second body member and configured to allow the guidewire to pass therethrough. According to the method, the guidewire may be passed through the second channel, and the second device may be slid along the guidewire to dispose the second device proximate the first device. At least one additional fastener may be applied to the second device via the at least one second opening of the second reinforcement member to affix the second device at the site of the injury or defect.

In some embodiments, the method may further comprise tensioning the guidewire and securing the guidewire externally or subcutaneously.

The features, functions, and advantages that have been discussed may be achieved independently in various embodiments of the present disclosure or may be combined with yet other embodiments, further details of which may be seen with reference to the following description and drawings.

### BRIEF DESCRIPTION OF THE DRAWINGS

Having thus described embodiments of the disclosure in general terms, reference will now be made to the accompanying drawings, wherein:

FIG. 1 illustrates a plan view of a device including a reinforcement member with grommets, in accordance with an embodiment of the disclosure;

FIG. 1A illustrates a perspective view of the reinforcement member of FIG. 1, in accordance with an embodiment of the disclosure;

FIG. 1B illustrates a cross-sectional view of the device of FIG. 1, in accordance with an embodiment of the disclosure;

FIG. 2 illustrates a plan view of a device including a reinforcement member with grommets and interconnection portions, in accordance with an embodiment of the disclosure;

FIG. 2A illustrates a perspective view of the reinforcement member of FIG. 2, in accordance with an embodiment of the disclosure;

FIG. 2B illustrates a cross-sectional view of the device of FIG. 2 at a grommet, in accordance with an embodiment of the disclosure;

FIG. 2C illustrates a cross-sectional view of the device of FIG. 2 at an interconnecting portion, in accordance with an embodiment of the disclosure;

FIG. 3 illustrates a perspective view of a device including a solid body member and a channel, in accordance with an embodiment of the disclosure;

FIG. 4 illustrates a perspective view of a device including a woven body member and a channel, in accordance with an embodiment of the disclosure;

FIG. 5 illustrates a perspective view of a device including pockets for supporting multiple reinforcement members, in accordance with an embodiment of the disclosure;

FIG. 6 illustrates a plan view of a device including attachment threads and attachment needle/anchoring fastener, in accordance with an embodiment of the disclosure;

FIG. 7 illustrates an injury or defect site with a device installed, in accordance with an embodiment of the disclosure;

FIG. 8 is a flowchart illustrating an example method for sternal/chest wall repair, in accordance with an embodiment of the disclosure; and

FIG. 9 illustrates an injury or defect site with a first device installed and additional devices being installed along a common guidewire, in accordance with an embodiment of the disclosure.

### DETAILED DESCRIPTION

Embodiments of the present invention will now be described more fully hereinafter with reference to the accompanying drawings in which some but not all embodiments of the inventions are shown. Indeed, these inventions may be embodied in many different forms and should not be construed as limited to the embodiments set forth herein; rather, these embodiments are provided so that this disclosure will satisfy applicable legal requirements. As used herein, the terms “inner,” “outer,” “top,” and “bottom” are used to describe the relative position of certain components or portions of components. The term “longitudinal” refers to a direction that extends from the top of a structure to the bottom. Similarly, the term “transverse” refers to a direction that extends from one side of the chest wall to the other, from left to right or vice versa. The term “lateral” refers to a location of an anatomical structure (such as a bone), a feature of the present invention, or movement in a direction that is farthest from the center of the transverse plane of the respective structure. In addition, the term “superior” is herein used as a relative term to refer to a location of an anatomical structure (such as a bone), a feature of the present invention, or movement that is closer to the top of the respective structure. Conversely, the term “inferior” is used as a relative term to refer to a location of an anatomical structure (such as a bone), a feature of the present invention, or movement that is farther from the top of the respective structure. As used herein, “device” and “endoplate” may be used interchangeably. Furthermore, although each example described herein refers to the closure of a dissected sternum, embodiments of the described invention may be used to hold together other bones in which a longitudinal fracture or cut exists. In some cases, for example, embodiments of the invention may be used to treat a pectus deformity.

As noted above, in one example, following thoracic surgery the chest wall may become unstable. In some cases, instability is due to bone that has not healed from thoracic surgery. According to conventional methods, a thoracic surgeon will attach metal plates to the sternum of the patient along either the transverse or longitudinal planes of the sternum in order to reinforce the reconstructed sternum and to further provide support and reinforcement of the chest wall. In some cases, the sternum may need to be reopened following thoracic surgery due to the occurrence of a second thoracic event.

A conventional metal plate may be composed of a series of repeating metal units with holes in their centers, wherein the holes are designed to accept a fastener. Each metal unit of the metal plate has an interconnection with the one or more other units of the metal plate. The metal plate may, for example, be composed of titanium, stainless steel, or nitinol. In some cases, the metal unit may stand separate and apart from other units as a single unit.

According to conventional methods, the thoracic surgeon will first insert a port or ports into the patient's body to allow access to the thoracic cavity. Then the metal plate will be delivered to the site within the chest wall. The metal plate is fixed to the sternum via fasteners. For example, the metal plate may be fixed to adjacent ribs and/or other tissue structures on one or both sides of the thoracic cavity. The sternum is comprised of three sections referred to as the manubrium (upmost section), the corpus (body), and the xiphoid process (lower end). The sternum is a bone centrally located within the thoracic cavity. The one or more metal plates may be fixed to the sternum along the entire length of the sternum or only a portion of the length. The metal plate or plates are fixed to the chest wall with metal screws. The surgeon will use as many metal plates as is needed to stabilize the chest wall, including a single metal plate or a plurality of metal plates fixed along the length of the sternum and/or to adjacent ribs and/or other tissue structures. The surgeon may select plates having different lengths and/or shapes based on the patient's particular anatomy, the configuration of the fracture to be repaired (e.g., the size, location, orientation, surrounding structures, etc. of the fracture), and/or other factors. In some cases, the surgeon may also wrap wire around a portion of the sternum in order to close the sternum along the incision.

The metal plates used in conventional techniques, however, are rigid, often lacking modularity and limiting fixation techniques. Such devices and methods may cause additional symptoms to be experienced by the patient, particularly in surgical procedures requiring tissue reinforcement or graft anchoring. Those symptoms may include pain when breathing or coughing, shortness of breath, or a generalized pain within the chest cavity. Further, conventional plates do not provide easy access to the thoracic cavity following a second thoracic event.

Accordingly, embodiments of the invention described herein offer adaptability, secure fixation, and biocompatibility by introducing a composite design that combines structural rigidity and material flexibility. As described below, embodiments provide for the reconstruction of the sternum and reinforcement of the chest wall using a device or endoplate that includes pre-formed holes for bone screw fixation, anatomical contouring, and mechanical stability. The endoplate may be a modular device for soft tissue or structural fixation, graft support, or reinforcement in medical applications. Embodiments provide a hybrid anchoring mechanism combining a rigid support plate with a flexible fabric interface and/or an integrated channel for multiple fixation options. In particular, embodiments of the invention provide for a device that includes a reinforcement member encased by a body member, where the body member may be made of a material that is pliable and configurable. For example, the body member may, in some embodiments, be polymer-based and may be woven (e.g., tightly-woven), knitted, and/or molded. According to embodiments of the invention, the device as described herein provides more flexibility, conformability, pliability, and strength than conventional metal plates. Embodiments of the device may be fixed to the sternum and/or adjacent ribs on one or both sides of the thoracic cavity and may also be fixed to other tissue structures to enhance and encourage healing, as described in greater detail below. In particular, the device may be able to be applied to the injured site (e.g., the site of the bone fracture or deformity to be repaired) in different planes and directions so as to address a complex topographic anatomy (e.g., a complex fracture of the chest wall with displacement) while still providing secure fixation and support at the injured site.

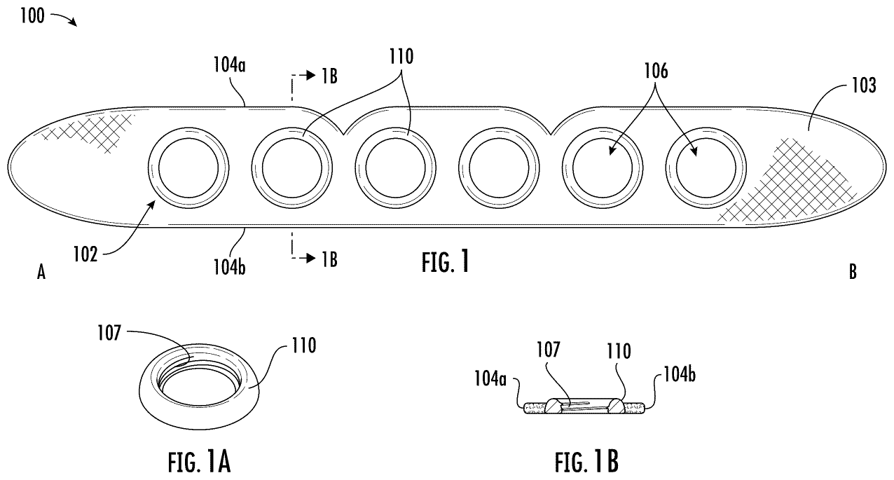

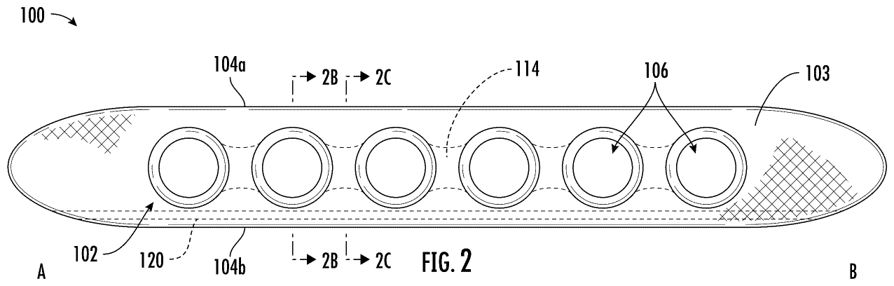

Turning to FIG. 1, in some embodiments, the device 100 may comprise a reinforcement member 102 and a body member 103 surrounding and supporting the reinforcement member. The reinforcement member 102 may define at least one opening 106, and each opening may be configured to receive a fastener so as to secure the device to a corresponding bone portion. As such, the device may be configured to be placed on an outer surface of bone proximate a fracture or location to be repaired, such that the device (or multiple cooperating devices) may be configured to stabilize the fracture or fractures. In this way, when the device or devices are installed, the fracture is substantially closed to allow healing to occur.

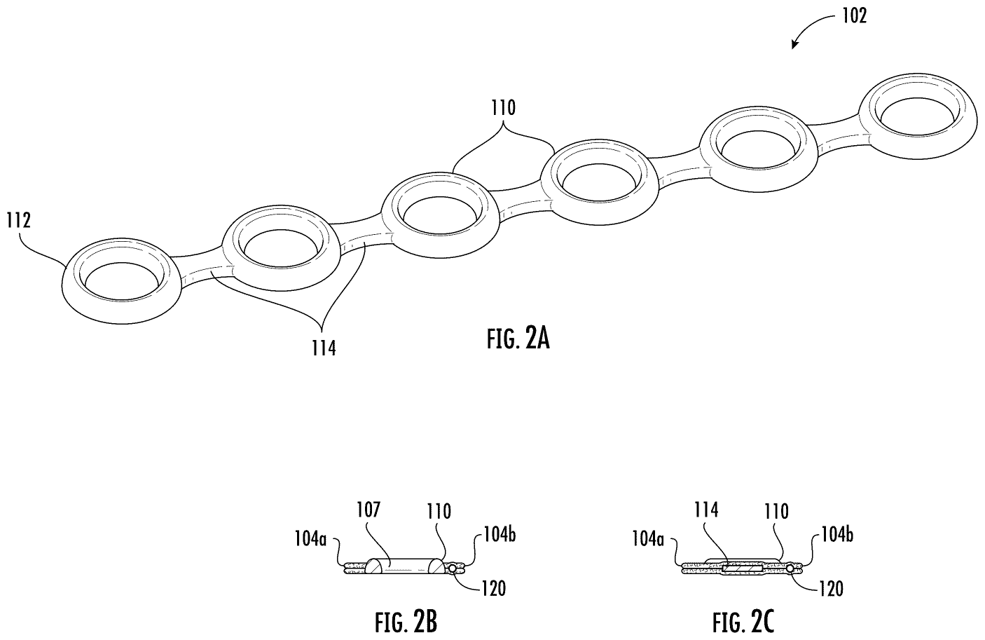

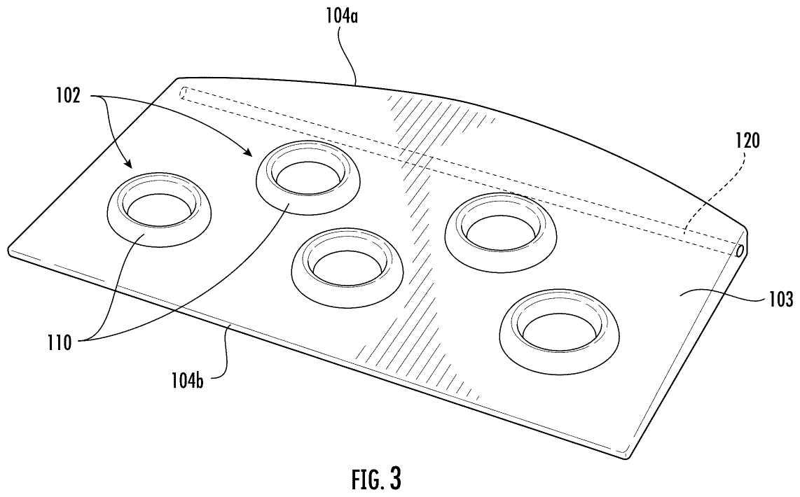

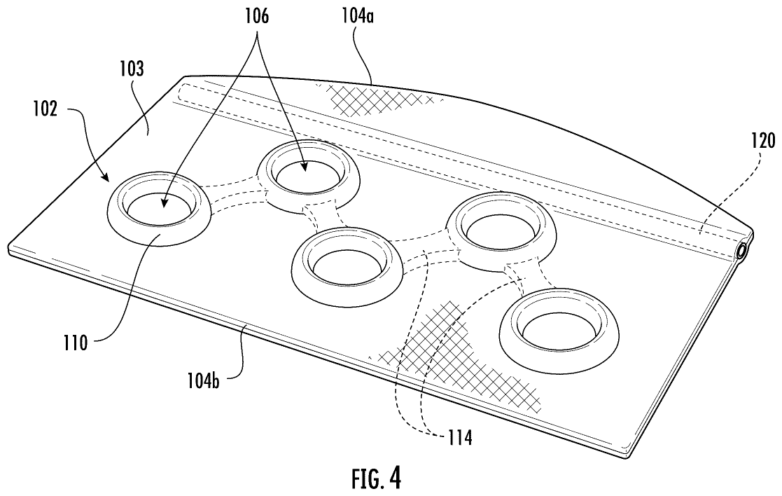

With reference to FIGS. 1-1B, in some embodiments, the reinforcement member 102 may comprise at least one grommet 110, but in some cases may include a plurality of grommets, such as 2, 3, 4, 5, 6, or as many as 40 grommets or more. In the depicted embodiment, each grommet 110 is a rigid annular member that, when applied to the body member 103, is configured to structurally reinforce a hole formed in the body member 103, thereby creating a reinforced opening 106 in the device 100, shown in FIG. 1. Each grommet 110 may be made of a biocompatible metal, such as a titanium alloy, stainless steel, nitinol, a high-performance biopolymer, such as polyetheretherketone (PEEK) or polyetherketoneketone (PEKK), or other biocompatible material that has been approved for long-term use in the human body. The grommets 110 may be arranged linearly, such as in the embodiments depicted in FIGS. 1 and 2, or, in other embodiments, may be arranged in a “z” or zig-zag formation, as depicted in FIGS. 3 and 4. Moreover, the grommets 110 may be evenly spaced in some embodiments, whereas in others the grommets may be unevenly spaced, such that the distance between some adjacent grommets may be different than the distance between other adjacent grommets.

In some embodiments, for example as shown in FIG. 2, the grommets 110 are interconnected to form a plate 112. In this regard, the plate 112 may comprise at least two grommets 110, with interconnecting portions 114 between adjacent grommets. The interconnecting portions 114 may be narrower at their narrowest dimension than a diameter of the grommets 110. In some embodiments, the interconnecting portions 114 may be integrally formed with the grommets 110 (e.g., made of the same material during the same manufacturing process), whereas in other embodiments the interconnecting portions 114 and the grommets 110 may be made of different material and/or may be separately formed and subsequently fixed to each other, such as via welding or adhesion. As described above, the grommets 110 may be arranged linearly, such that the plate 112 is substantially straight, while in other embodiments the grommets 110 may be arranged in a “z” or zig-zag formation, such that the plate 112 has a “z” or zig-zag shape. Moreover, the grommets 110 may be evenly spaced in some embodiments, whereas in others the grommets may be unevenly spaced, such that the distance between some adjacent grommets may be different than the distance between other adjacent grommets.

In some embodiments, the reinforcement member 102 may define an inner surface, and the inner surface may define threads configured to engage corresponding threads on a fastener (e.g., a fastener configured to be applied to the opening 106 to secure the device 100 to bone). For example, an inner surface 111 of the grommets 110, shown in FIG. 1A, may define the threads in some embodiments.

Turning again to FIGS. 1-2B, the body member 103 may, in some embodiments, be made of a tightly-woven material, such as individual fibrous threads that are interwoven to form the body member. For example, the body member 103 may comprise tightly-woven fibrous threads made of at least one of polytetrafluoroethylene (cPTFE), expanded polyethylene (ePE), decellularized fish skin, acellular dermal material, biocellular material, collagen, porcine small intestinal submucosa (SIS), or other biocompatible material that has been approved for long-term use in the human body. Examples may include Gore-Tex®, Kerecis®, Arthroflex®, or similar biological/engineered scaffold materials. These threads may be interwoven to form the shape of the body member 103, where the overall shape of the body member is configured to encompass (e.g., surround, support, and/or house) the reinforcement member 102, as described in greater detail below. In this way, the body member 103 may be configured to provide a soft-tissue-facing surface that promotes cellular integration, soft tissue support, and/or graft fixation. As such, the body member 103 may be woven or otherwise formed to define holes configured to align with and correspond to the holes defined by the grommets 110, such that when the reinforcement member 102 is positioned within the body member 103, the holes of the body member are reinforced to create the openings 106 of the reinforcement member 102 and overall device. In some embodiments, however, the material or threads forming the body member 103 may be configured to separate upon application of a force (e.g., force applied by a tip of a fastener) to form a hole allowing the fastener to pass therethrough, such that the hole may not be pre-formed.

In some embodiments, the grommets 110 of the reinforcement member 102 may be configured to be aligned with and engage corresponding holes in the body member 103. For example, each grommet 110 may be configured to engage a corresponding hole in the body member 103 via a press fit, a snap fit, adhesive, or other technique. In some cases, the grommets 110 may include a groove on an outer surface of the grommet configured such that the fabric edge of the hole fits into and is held by the groove. In still other cases, the grommets may be configured in two halves, with the halves configured to snap together and be held in place about the corresponding hole via a mechanical snap fit or press fit.

In some embodiments, each of the plurality of fibrous threads forming the body member 103 may extend from one end A of the device 100 to the other end B. In other embodiments, only a portion of the threads may extend the length of the device 100 from one end A to the other end B, with additional threads interwoven or otherwise incorporated into the body member 103 at predetermined locations to form the desired shape.

The body member 103 may be configured to have various shapes and sizes, such that it may accommodate various sizes and configurations of reinforcement members 102 (e.g., reinforcement members with one grommet, two grommets, three grommets, four grommets, etc., as well as grommets of different diameters). In some embodiments, the body member 103 may comprise elongated ends A, B. In still other embodiments, at least one edge of the body member 103 is contoured. For example, the body member 103 may define a first longitudinal edge 104a and a second longitudinal edge 104b opposite the first longitudinal edge. In some embodiments, such as the embodiment depicted in FIG. 2, the first longitudinal edge 104a and the second longitudinal edge 104b may be substantially similar in shape, such that the device 100 is symmetrical across a longitudinal axis. In other embodiments, however, such as depicted in FIG. 1, one of the first longitudinal edge 104a and the second longitudinal edge 104b may be contoured with respect to the other edge. For example, one of the edges 104a, 104b may be substantially linear (e.g., straight line), whereas the other of the edges 104a, 104b may be curved, scalloped, or otherwise contoured, as shown in the example of FIG. 1. Moreover, the edges 104a, 104b may have the same configuration in some cases (e.g., both straight or both contoured) or may have different configurations. In the example depicted in FIG. 1, the first longitudinal edge 104a is scalloped, whereas the second longitudinal edge 104b is a relatively straight line.

In some embodiments, because the body member 103 is tightly-woven, and/or depending on the rigidity of the material selected for the body member, the body member may be configured to be shaped by the surgeon or practitioner for a particular procedure. In other words, the body member 103 may be sufficiently pliable, and at the same time sufficiently stiff, such that the surgeon or practitioner may bend and form the overall shape of the body member to conform to the anatomical topology of the site to which the device will be applied. This may include shaping the device 100 in one, two, or three planes. As one example, the body member 103 may be curved such that the device 100 takes on a circular shape. As another example, the body member 103 may be twisted or bent. In this way, the device 100 may be customized to match the natural orientation of ribs and intercostal spaces to allow for rib plating, sternal bridging, or extended anterior chest wall reconstruction.

In some embodiments, the body member 103 and/or the reinforcement members 102, or portions thereof, may be composed of biodegradable materials. In such cases, the biodegradable materials are designed to degrade over time within the patient's body. In some cases, the woven material forming the body member 103 may be single- or double-layered. For example, in the embodiment depicted in FIGS. 1-1B, the body member 103 comprises a single layer. In the embodiment depicted in FIGS. 2-2C, however, the body member 103 comprises two layers, such that the interconnecting portions 114 are received between the two layers, as shown in FIG. 2C. In some embodiments, the body member 103 may comprise thin layers of material that result in a lower profile, causing less protrusion of the device overall and greater flexibility. As noted above, greater flexibility and pliability of the device 100 in turn results in greater conformity of the device to the sternum and/or adjacent bone or tissue structures, decreasing pain, discomfort, and other symptoms that may otherwise be experienced by the patient.

In some embodiments (e.g., best shown in FIGS. 2, 3, and 4) the body member 103 may comprise a channel 120 proximate an edge of the body member (e.g., proximate the first longitudinal edge 104a, as depicted). The channel 120 may be configured to allow a wire (e.g., a guidewire) to be passed therethrough. For example, in some embodiments, the diameter of the channel 120 may range from approximately 0.5 mm to approximately 5 mm. In this way, the surgeon or other practitioner may be able to use the guidewire to insert the device 100 into the human body and dispose the device at the inured site, such as according to conventional or novel methods. In addition or alternatively, the channel 120 may be used to enable the passage of fixation means, such as a suture, wire, or surgical (flat) tape.

With reference now to FIG. 3, in some embodiments, the reinforcement member 102 may be integral to the body member 103. For example, the reinforcement member 102 and the body member 103 may be formed of the same material during the same manufacturing process, thereby creating a singular structure. The reinforcement member 102 and the body member 103 may, in such embodiments, be integrally formed of at least one of ePTFE, ePE, decellularized fish skin, acellular dermal material, biocellular material, collagen, a porcine small intestinal submucosa (SIS), a titanium alloy, or a high-performance biopolymer, such as PEEK or PEKK. In such embodiments, the body member 103 may be configured such that a thickness of the body member decreases from the first longitudinal edge 104a toward the second longitudinal edge 104b. For example, in some embodiments, a thickness of the body member 103 at the first longitudinal edge 104a is approximately 5 mm to approximately 20 mm, whereas a thickness of the body member at the second longitudinal edge 104b is approximately 1 mm to approximately 15 mm. Moreover, the larger thickness of the material at the first longitudinal edge 104a may be selected to accommodate the channel 120. The thickness of the body member 103, with or without the taper, may generally range from approximately 1 mm to approximately 20 mm, depending on the particular injury or defect to be addressed, the patient's anatomy, and other considerations.

In some cases, the body member 103 may define the channel 120 proximate the first longitudinal edge 104a, and the channel may be configured to allow a wire to pass therethrough. Depending on the configuration of the body member 103 (e.g., whether it is woven, knit, molded, etc.) and the material from which it is made (e.g., metal or biopolymer), the channel 120 may be formed integrally with the body member and/or embedded in the body member, such as shown in FIG. 3. For example, as shown in FIG. 3, where the body member 103 is made of a solid material such as metal or a biopolymer, the channel 120 may be formed as a tubular void in the material. As another example, where the body member 103 is made of a woven or knit material, the channel 120 may be formed via walls of the body member that are sewn or adhere so as to create an open-ended compartment or pathway through the layers of woven or knit fabric. In other embodiments, however, the channel 120 may be a tube that is attached to the body member, as shown in FIG. 4 where the body member is woven. In this case, in which the body member 103 is woven, the channel 120 may comprise a metal or biopolymer tube that is attached (e.g., sewn, adhered, etc.) within the body member proximate the first longitudinal edge 104a or the second longitudinal edge 104b. The channel 120 may, in some cases, mirror the curvature or shape of the corresponding edge of the body member 103 and/or the reinforcement member 102 supported thereby.

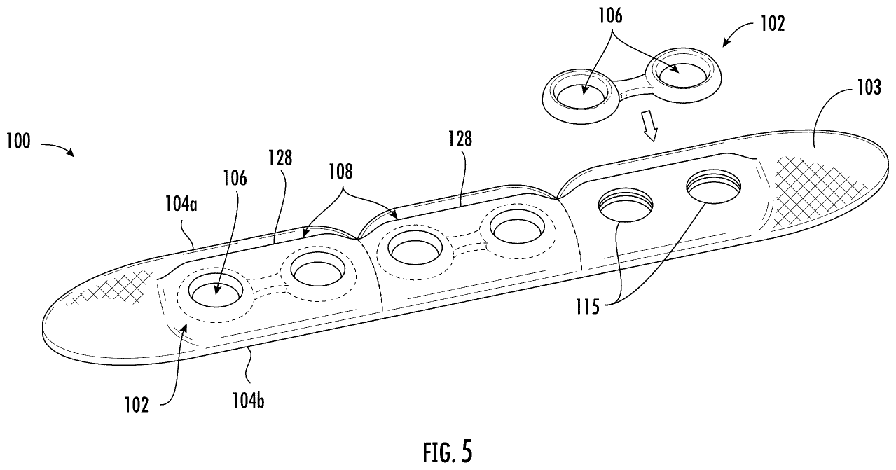

With reference now to FIG. 5, in some embodiments, the body member 103 may comprise one or more pockets 108 or sleeves, each pocket being configured (e.g., sized and shaped) to receive a reinforcement member 102 therein. In the depicted embodiment, for example, one or more apertures 128 providing access to the pockets 108 may be formed in a respective longitudinal edge 104a, 104b of the device 100, such that a reinforcement member 102 may be slipped into the pocket 108 via the aperture(s) 128 and positioned within the body member 103. The material forming the pocket 108 may likewise define holes 115 configured to align with the openings 106 of the reinforcement members 102, once received within the pocket. In some embodiments, the pocket 108 may have a length (e.g., at the aperture 128) of approximately 2-8 cm, a width of approximately 2-16 mm, and a thickness of approximately 2-8 mm. After the reinforcement member 102 is received within the pocket 108, the aperture 128 may be stitched closed or otherwise sealed to enclose the reinforcement member 102 within the pocket.

In some embodiments, the body member 103 may comprise only one pocket 108 configured to receive a single reinforcement member 102, such as a reinforcement member that includes multiple grommets 110 forming multiple openings 106 for receiving fasteners. In other embodiments, however, the body member 103 may comprise more than one pocket 108 and more than one aperture 128 for providing access to each corresponding pocket, where each pocket is configured to receive one reinforcement member 102, as shown in FIG. 5. According to such embodiments, the device 100 may thus comprise multiple reinforcement members 102, wherein each reinforcement member defines multiple openings 106. For example, as depicted in FIG. 5, the body member 103 may include three pockets 108 and three apertures 128 configured to receive three reinforcement members 102. Each reinforcement member 102 may, according to the depicted embodiment, include two grommets 110 forming two openings 106, such that the device 100 depicted in FIG. 5 has three reinforcement members 102 having two openings 106 each to provide the illustrated six-opening configuration. In some further embodiments, in which multiple reinforcement members 102 are supported by a single body member 103, the reinforcement members may not be identical. For example, the openings 106 in one reinforcement member 102 may be larger than the openings in another reinforcement member, one reinforcement member may have openings whereas another in the same body member may not, or the configuration of the openings (e.g., linear versus non-linear) may differ. Moreover, other characteristics of the reinforcement members, such as the thickness, rigidity, material, size, etc., may also be different. In some embodiments, the body member 103 may define or include a channel configured similarly to the channel 120 described above.

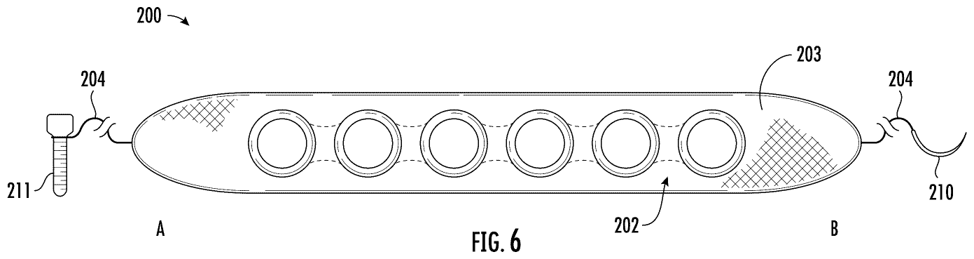

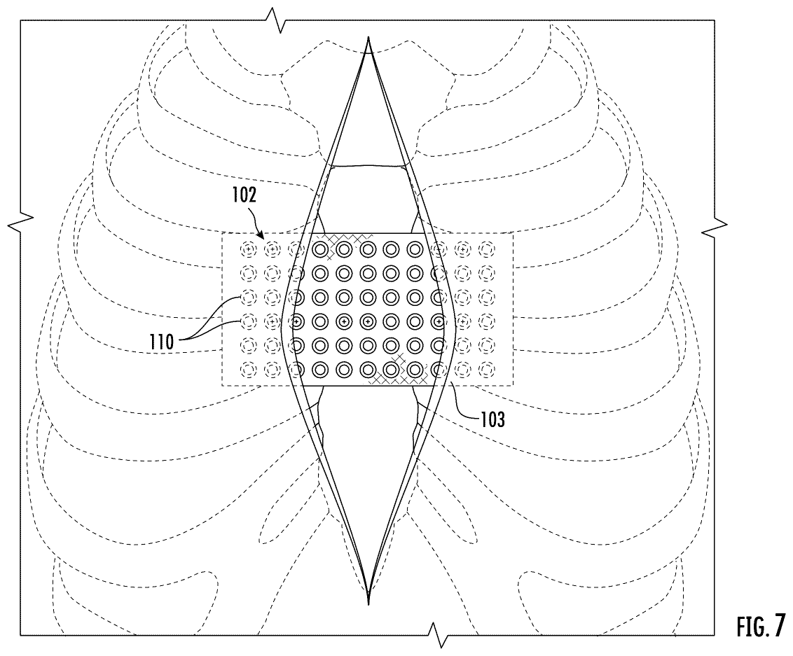

In some embodiments, as shown in FIG. 6, a device 200 is provided that comprises one or more attachment threads 204 and/or an attachment needle 210 at one or both ends A, B of the body member 203. For example, as shown in FIG. 6, the device 200 may comprise an attachment thread 204 extending from both ends of the body member 203, wherein the attachment thread is one of the plurality of the threads that are interwoven to form the body member. In this regard, one of the plurality of threads forming the body member 203 may be longer than the other threads, such that it extends beyond the ends A, B of the device.

Moreover, in some embodiments, an attachment needle 210 may be fixed to the end of one or more of the attachment threads 204, while an anchoring fastener, such as a screw, clip, staple, etc. may be fixed to the end of the other attachment thread. In the depicted embodiment, for example, the device 200 comprises two attachment threads 204, one at each end A, B of the device, where one attachment thread 204 comprises an attachment needle 210 and the other attachment thread comprises a screw 211 for anchoring the device 200 to the bone. In other embodiments, however, more than one attachment thread may be provided at one or both ends, or one of the ends may not include any attachment threads. In addition, attachment needles 210 and/or anchoring fasteners (e.g., screws 211) may be provided on all or fewer than all attachment threads 204 at one or both ends, or none of the attachment threads may include attachment needles and/or anchoring fasteners. The attachment needles 210 may be configured to pierce soft tissue, for example, to allow the attachment thread(s) 204 to be wrapped around bone, thereby securing the respective end A, B of the device 100 in place within the thoracic cavity.

For example, the device 200 shown in FIG. 6 may be secured to a portion of a patient's sternum along the transverse plane within the chest cavity. The attachment thread 204 and/or attachment needle 210 of the device 200 may be wrapped around one or more ribs located on one or both sides of the chest wall in the location requiring fixation to secure the device in place and further stabilize the closure of the sternum. In some embodiments, the thread is wrapped around a first lateral edge of a first portion of the sternum, around the back of the sternum and around to the lateral edge of a second portion of the sternum to close the dissected portions of the sternum along the longitudinal plane.

With reference again to FIGS. 1-5, embodiments of the device 100 may be used to provide minimally invasive chest wall stabilization according to conventional surgical techniques as well as novel techniques. For example, in such cases in which attachment threads and attachment needles/anchoring fasteners are not integral to the device 100, conventional suturing tools, including needle and thread, staples, screws, and/or other fasteners may be used to secure the device in place.

In some embodiments, the body member 103 may be approximately 1-300 mm wide and approximately 1-300 mm long. For example, with reference to FIG. 7, in some embodiments, the body member 103 may be configured in the form of a sheet having a plurality of holes with a plurality of grommets 110 reinforcing those holes. As depicted, in some cases the grommets 110 forming the reinforcement member may be arranged in a grid-like configuration. In this way, the device can be tailored (e.g., cut and/or trimmed) to match the chest wall defect for longitudinal or transverse placement. Certain grommets 110 may be selected for insertion of fasteners to secure the device in place at the appropriate location, depending on the configuration of the defect (e.g., the location of the bones, tissue, etc. to be repaired). One skilled in the art in view of this disclosure would recognize that the various features and components described herein can be included or excluded in various devices and combined in different ways. For example, in some cases, the sheet-like body member 103 shown in FIG. 7 may not include individual grommets 110 but may instead include one or more pockets (such as the pockets 108 of FIG. 5) that are configured to receive corresponding reinforcement members 102.

Various techniques may be used to apply one or more devices 100, 200 to the injured site in a patient's body, such as for pectus excavatum and complex chest wall repair. In some embodiments, for example, a surgeon may use an open approach (e.g., a Modified Ravitch or Sternotomy Adjunct approach); an endoscopic approach (e.g., thoracoscopic or robotic); or a hybrid approach that combines both an open approach and an endoscopic approach. For example, an open approach may be used to address severe deformities or reoperations or when full exposure is needed. An endoscopic approach may be used in the case of minimally invasive pectus repair, flail chest, or trauma. A hybrid approach may be used to address asymmetric deformities or revision cases or when bar placement alone is insufficient.

Using any of these techniques, a guidewire (e.g., a wire, tape, etc.) may be inserted through an incision or other access opening in the patient's body and anchored in a distal location proximate the site in the patient's body to be repaired. The free end of the guidewire may then be threaded through the channel 120 of the selected device 100, 200, and the device may be moved along the wire to the injured site. The device 100, 200 may then be secured to the sternum with one or more fasteners (not shown) via the openings 106, 206. In some embodiments, the fasteners may be metal screws designed to bore into the bone without affecting surrounding organs and other tissues. The fasteners may be configured (e.g., sized and shaped) to fit within the openings 106, 206 of the reinforcement members 102, 202. In such embodiments, the fasteners may be inserted in all of the provided openings 106, 206 or only in some of the openings, depending on the location of the device within the chest cavity and as determined by the surgeon based on the needs of the patient and the condition being addressed. Additionally or alternatively, the device 100, 200 may be fixed to either side of the chest wall using staples, sutures, or zip ties. The device 100, 200 may be fastened to the sternum itself and/or to ribs and/or other tissue structures located on either side of the chest cavity. In some embodiments, and as described in greater detail below, one or more devices 100, 200 may be secured to the sternum and/or ribs along the transverse plane of the chest wall.

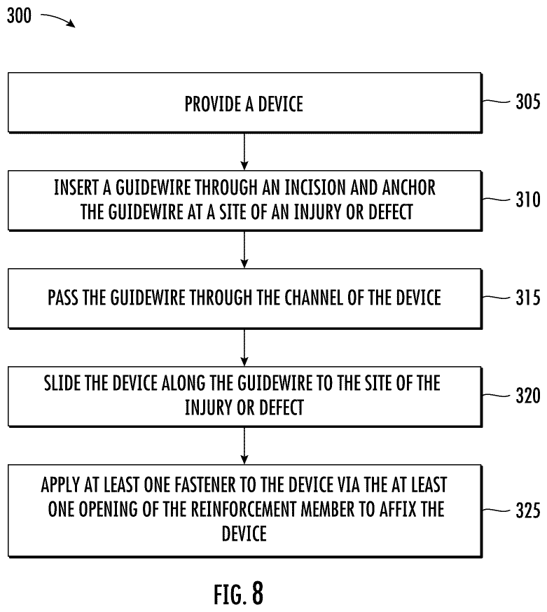

Referring to FIG. 8, embodiments of a method 300 for repair of an injured site is described. According to example embodiments, the technique provides guided telescopic delivery of the device to stabilize the chest wall in a way that allows internal-external tensioning, modular fixation, and precise anatomical alignment that is adaptable to any access strategy, as noted above. In some embodiments, the method 300 initially provides a device comprising a reinforcement member (such as the reinforcement member 102 described above in connection with the figures) and a body member at Block 305. The reinforcement member may define at least one opening. The body member may surround and support the reinforcement member and may comprise a channel proximate an edge of the body member, where the channel is configured to allow a wire to pass therethrough, as described above. At Block 310, a guidewire (e.g., wire or tape) may be inserted through an incision, and the guidewire may be anchored at the site to be repaired. In some cases, the device may be guided over a pre-positioned guidewire (e.g., a suture, tape, etc.) without rotating or torquing during insertion. Moreover, longitudinal tensioning through the channel may allow distributed pull across the device, reducing stress concentration, and the guidewire may remain coaxial with the direction of the rib or sternum contour, reducing shear forces and abrasion.

The wire may be passed through the channel of the body member at Block 315, and the device may be slid along the wire to the injured site at Block 320. At least one fastener may be applied to the device via the at least one opening of the reinforcement member to affix the device at the injured site at Block 325. In some cases, the fasteners (e.g., screws) may be pre-mounted to the device, such that the device is delivered to the site of the injury or defect with the fastener already in the opening of the reinforcement member, ready to be secured to the bone at the proper location.

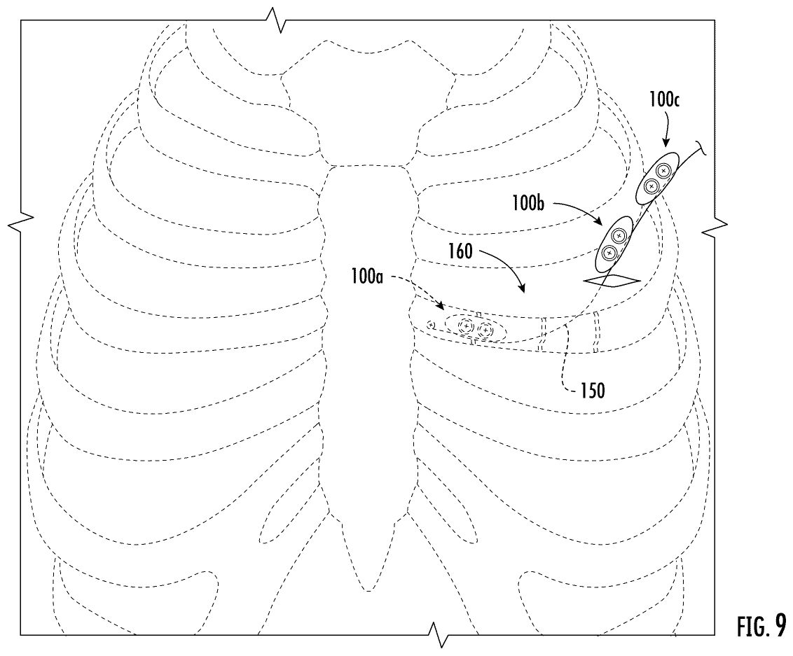

With reference to FIG. 9, in some embodiments, multiple devices 100 may be used in series to address an injury 160 or defect in the chest wall, such as to provide linear or segmental fixation of the chest wall. In such embodiments, the device 100 may be a first device 100a, and the method may further include providing a second device 100b. Accordingly, the reinforcement member 102 may be a first reinforcement member, and the body member 103 may be a first body member. The opening 106 may be a first opening, and the channel 120 may be a first channel. The second device 100b may be configured similarly to the first device 100a and may comprise a second reinforcement member, where a second body member surrounds and supports the second reinforcement member. The second reinforcement member may define at least one second opening, and the second body member may comprise a second channel proximate an edge of the body member that is configured to allow the guidewire 150 (which also passes through the first channel of the first device) to pass therethrough. The second device 100b may then be moved to the injury site 160 by sliding the second device 100b along the guidewire 150 to dispose the second device proximate the first device 100a. At least one additional fastener may then be applied to the second device 100b via the at least one second opening of the second reinforcement member to affix the second device at the site of the injury or defect.

Likewise, if needed, a similarly-configured third device 100c may be delivered to the injury site 160 via the same guidewire 150 and appropriately applied to repair the injury or defect, and, if needed, additional devices may similarly be used. In the example depicted in FIG. 9, three devices 100a, 100b, 100c are being applied to repair the injury or defect 160; however, four, five, six, or more devices may be used, depending on the patient's anatomy, the injury/defect being addressed, and other considerations, as determined by the surgeon or other practitioner. As noted above, the multiple devices may be similarly configured (e.g., sized and shaped) or may be different from each other, such as when one device is larger or smaller than another, has a different shape (e.g., linear versus non-linear), has a different number or arrangement of openings in the reinforcement member, etc.

In some embodiments, the method of repair may further comprise tensioning the guidewire 150 after the guidewire has been sufficiently anchored proximate the injury site 160 and secured externally or subcutaneously, as would be recognized by one skilled in the art in view of this disclosure. Once the devices 100a, 100b, 100c have been secured to the bone via fasteners (e.g., screws) through the openings, the free end of the guidewire may be trimmed and/or tightened, such as via a Cor-knot® solution, as would be understood by those skilled in the art in view of this disclosure. In some embodiments the method 300 and/or other methods described herein may include additional steps, such as any single step or any combination of steps described herein. Although FIG. 8 shows example blocks of the method 300, in some embodiments, the method may include additional blocks, fewer blocks, different blocks, or differently arranged blocks than those depicted in FIG. 8. Additionally, or alternatively, two or more of the blocks of method 300 may be performed in parallel.

Embodiments of the device and associated methods are therefore described for providing a modular system for sternal/chest wall repair with multiple optional access routes. Guided delivery promotes anatomical alignment, and dual fixation (fasteners and guidewire) enable the device (or devices) to resist migration and rotation. Through the use of multiple devices along a single guidewire, as described above, the system may be expandable, allowing a practitioner to add devices along the same rib or ribs (for example). Moreover, the body member interface promotes integration into the patient's body and reduces irritation, making the device tissue-friendly and allowing the device to be used both inside and outside the chest wall, as needed.

Although many embodiments of the present disclosure have just been described above, the present disclosure may be embodied in many different forms and should not be construed as limited to the embodiments set forth herein; rather, these embodiments are provided so that this disclosure will satisfy applicable legal requirements. Also, it will be understood that, where possible, any of the advantages, features, functions, devices, and/or operational aspects of any of the embodiments of the present disclosure described and/or contemplated herein may be included in any of the other embodiments of the present disclosure described and/or contemplated herein, and/or vice versa. For example, although embodiments of the invention described herein are described in the context of thoracic surgery, in other embodiments the devices, components, and/or methods described herein may be used in maxillofacial surgery, orthopedic surgery, cardiovascular surgery, and/or thoracic surgery. Moreover, the sizes, shapes, configurations, and materials may be modified according to the type of surgery to be performed, the location in the patient's body to be addressed, preexisting conditions of the patients, and/or other considerations for the surgery. Although certain examples of fasteners for securing the device are described above, embodiments of the invention may be secured by any form of fastening method including staples, screws, ties, zip ties, and/or adhesive, including synthetic and bioengineered adhesives.

While certain exemplary embodiments have been described and shown in the accompanying drawings, it is to be understood that such embodiments are merely illustrative of and not restrictive on the broad disclosure and that this disclosure is not to be limited to the specific constructions and arrangements shown and described, as various other changes, combinations, omissions, modifications and substitutions, in addition to those set forth in the above paragraphs, are possible. In light of this disclosure, those skilled in the art will appreciate that various adaptations, modifications, and combinations of the just described embodiments may be configured without departing from the scope and spirit of the disclosure. Therefore, it is to be understood that the disclosure may be practiced other than as specifically described herein.

What is claimed is:

1. A device comprising:
a reinforcement member, wherein the reinforcement member defines at least one opening; and
a body member surrounding and supporting the reinforcement member,
wherein the device is configured to be placed on an outer surface of bone proximate an injury or defect,
wherein each opening of the reinforcement member is configured to receive a fastener so as to secure the device to the bone, and
wherein, when installed, the injury or defect is substantially stabilized to allow healing to occur.

2. The device of claim 1, wherein the reinforcement member comprises a plurality of grommets.

3. The device of claim 2, wherein an inner surface of at least one grommet defines threads configured to engage corresponding threads on the fastener.

4. The device of claim 2, wherein the grommets are arranged linearly.

5. The device of claim 2, wherein the grommets are arranged in a z-formation.

6. The device of claim 2, wherein the grommets are interconnected to form a plate.

7. The device of claim 1, wherein the body member is a woven material.

8. The device of claim 1, wherein the body member comprises a channel proximate an edge of the body member and configured to allow a guidewire to pass therethrough.

9. The device of claim 8, wherein the channel is a tube attached to the body member.

10. The device of claim 8, wherein the channel is a compartment formed by the body member.

11. The device of claim 1, wherein the body member comprises elongated ends.

12. The device of claim 1, wherein the reinforcement member is integral to the body member.

13. The device of claim 1, wherein the body member defines a first longitudinal edge and a second longitudinal edge opposite the first longitudinal edge, wherein a thickness of the body member decreases from the first longitudinal edge toward the second longitudinal edge.

14. The device of claim 13, wherein the body member defines a channel proximate the first longitudinal edge configured to allow a wire to pass therethrough.

15. The device of claim 1, wherein the reinforcement member comprises at least one of metal, a bio-polymer, polyetheretherketone (PEEK), or polyetherketoneketone (PEKK).

16. The device of claim 1, wherein the body member comprises at least one of polytetrafluoroethylene (ePTFE), expanded polyethylene (ePE), decellularized fish skin, acellular dermal material, biocellular material, collagen, or porcine small intestinal submucosa (SIS).

17. The device of claim 1, wherein at least one edge of the body member is contoured.

18. A method for chest wall repair, the method comprising:
providing a device comprising a reinforcement member and a body member surrounding and supporting the reinforcement member, wherein the reinforcement member defines at least one opening, and wherein the body member comprises a channel proximate an edge of the body member and configured to allow a guidewire to pass therethrough;
inserting the guidewire through an incision and anchoring the guidewire at a site of an injury or defect;
passing the guidewire through the channel;
sliding the device along the guidewire to the site of the injury or defect; and
applying at least one fastener to the device via the at least one opening of the reinforcement member to affix the device at the site of the injury or defect.

19. The method of claim 18, wherein the device is a first device, the reinforcement member is a first reinforcement member, the body member is a first body member, the opening is a first opening, and the channel is a first channel, the method further comprising:
providing a second device comprising a second reinforcement member, wherein the second reinforcement member defines at least one second opening, and a second body member surrounding and supporting the second reinforcement member, wherein the second body member comprises a second channel proximate an edge of the second body member and configured to allow the guidewire to pass therethrough;
passing the guidewire through the second channel;
sliding the second device along the guidewire to dispose the second device proximate the first device; and
applying at least one additional fastener to the second device via the at least one second opening of the second reinforcement member to affix the second device at the site of the injury or defect.

20. The method of claim 18 further comprising:
tensioning the guidewire; and
securing the guidewire externally or subcutaneously.
